(19) United States
(12) Patent Application Publication
(10) Pub. No.: US 2003/0152910 A1
(43) Pub. Date: Aug. 14, 2003

(54) Method for identification and quantification of kinase inhibitors

(76) Inventors: Matthias Stein-Gerlach, Munich (DE); Helmut Mett, Graefelfing (DE); Manfred Marschall, Baldham (DE); Thomas Stamminger, Oberasbach (DE)

Correspondence Address: Leon R Yankwich, Yankwich & Associates, 201 Broadway, Cambridge, MA, 02139, US

(21) Appl. No.: 10/149,823

(22) PCT Filed: Dec. 6, 2000
(86) PCT No.: PCT/EP00/12303

(30) Foreign Application Priority Data
Dec. 6, 1999 (EP) 99 124 342.9

(51) Int. Cl. C12Q 1/00; C12Q 1/70; C12Q 1/68; C12N 15/86; G01N 33/53

(52) U.S. Cl. 435/4; 435/6; 435/5; 435/456; 435/7.1

(57) ABSTRACT

The present invention relates to a method for the identification of kinase inhibitors which is suitable for high-throughput screening. Moreover, the inhibitory effect of test substances can be quantified and a potential cytotoxicity of the respective inhibitors can be detected. The method is particularly suitable for the identification of inhibitors of viral kinases, e.g. herpes viral kinases.

The present invention relates to a method for the identification of kinase inhibitors which is suitable for high-throughput screening. Moreover, the inhibitory effect of test substances can be quantified and a potential cytotoxicity of the respective inhibitors can be detected. The method is particularly suitable for the identification of inhibitors of viral kinases, e.g. herpes viral kinases.

We developed a method that allows a rapid identification of inhibitors of human cytomegalovirus UL97 protein kinase activity or the activity of other kinases. The principle of this method is based on the observation that specific inhibitors of UL97 protein kinase activity are also able to antagonize ganciclovir (GCV) monophosphorylation which is catalyzed by UL97. Since GCV monophosphorylation leads to an accumulation of the cytotoxic product GCV-PPP within cells, cell death is prevented by specific UL97 kinase inhibitors. This principle is also true for any other kinase that is able to convert an inactive substrate into an active cytotoxic drug. Cell death can be quantitated by various methods: e.g. the colour conversion of the medium (containing a pH indicator) can be determined photometrically; alternatively, LDH activity within the cell layer can be taken as a measurement of the residual viable cells which inversely correlates with cell death. Within the same assay, by using a kinase inactive mutant, the target specificity and/or cytotoxicity of the used substance can be determined. Therefore, this assay allows for an extremely simple determination of the kinase inhibitory activity of substances together with a determination of cytotoxic effects exerted by the same substances, and is thus useful for the identification of novel therapeutic agents.

Thus, the present invention refers to a method for the identification of kinase inhibitors comprising the steps:

(a) providing a target cell comprising a nucleic acid encoding a kinase,

(b) adding to the target cell a substrate wherein the substrate is capable of being phosphorylated by said kinase and wherein said phosphorylated substrate is deleterious for said target cell,

(c) adding to the target cell at least one test compound and

(d) determining if said test compound is capable of at least partially inhibiting the deleterious effect of said phosphorylated substrate.

Further, the present invention refers to a method for the identification of kinase inhibitors comprising the steps:

(a) providing a target cell comprising a nucleic acid encoding a kinase,

(b) adding to the target cell a substrate wherein said substrate is capable of being phosphorylated by said kinase and wherein said phosphorylated substrate is deleterious for said target cell,

(c) adding to the target cell at least one test compound and

(d) determining, if said test compound is capable of at least partially inhibiting the phosphorylation of the substrate.

The methods according to the present invention allow the identification of inhibitors of any kinase which is able to convert a substrate into a product which is deleterious, e.g. cytotoxic for a target cell.

The kinase may be homologous for the target cell which is preferably a cultured eukaryotic cell, particularly a mammalian cell and more particularly a human cell, e.g. the human embryonic kidney cell 293 (ATCC CRL-15173). Preferably, however, an assay system is used, wherein the kinase is heterologous for said target cell. The introduction of a heterologous kinase gene into a target cell may be accomplished by transforming or transfecting said target cell with a vector comprising a nucleic acid encoding the kinase to be tested. Alternatively, a target cell may be used, which is infected by a virus carrying the nucleic acid encoding the kinase to be tested.

Preferably, the kinase is derived from a pathogen, particularly a microbial pathogen such as a bacterium, a unicellular eukaryotic organism or a virus. More preferably the kinase is a viral kinase, e.g. a herpes viral kinase.

The herpesviruses may be selected from human herpesviruses and herpesviruses from other mammals, such as bovine, equine, porcine and pongine herpesviruses. Suitable herpesviruses are selected from α-herpesviruses, e.g. simplexviruses such as herpes simplex virus 1, herpes simplex virus 2, bovine herpesvirus 2, cercopithecine herpesvirus 1 or varicellaviruses such as varicella zoster virus, porcine herpesvirus 1 (pseudorabiesvirus) bovine herpesvirus 1 and equine herpesvirus 1 (equine abortion virus). Further, the herpesvirus may be selected from β-herpesviruses, e.g. cytomegaloviruses such as human cytomegalovirus and from roseoloviruses, such as human herpesvirus 6, human herpesvirus 7 or aotine herpesviruses 1 and 3. Further, the herpesviruses may be selected from γ-herpesviruses, e.g. from lymphocryptoviruses such as Epstein-Barr virus, cercopithecine herpesvirus 2 or porcine herpesvirus 1, or from rhadinoviruses such as human herpesvirus 8, ateline herpesvirus 2 or saimiriine herpesvirus 1, or preferably, the virus is selected from human herpesvirus 1 (HSV-1), varicella zoster virus (VZV) or human cytomegalovirus (HCMV).

Preferably, the viral kinase is selected from human CMV UL97 kinase, human HSV-1 or -2 UL13 kinase, human VZV ORF47 kinase, human HHV-6 U69 kinase, human EBV BGLF4 kinase, human HHV-8 ORF36 kinase or kinases homologous thereto.

The viral kinase may be encoded by:

(a) the nucleic acid sequence as shown in SEQ ID No. 1, SEQ ID No. 3, or SEQ ID No. 5.

(b) a nucleic acid sequence corresponding to a sequence (a) in the scope of degeneracy of the genetic code, or

(c) a nucleic acid sequence hybridizing under stringent conditions with a nucleic acid of (a) or (b).

The nucleic acid sequence of the CMV UL97 kinase gene and the corresponding amino acid are shown in SEQ ID No. 1 and 2. The nucleic acid sequence of the HSV-1 UL13 kinase gene and the amino acid sequence corresponding thereto are shown in SEQ ID No.3 and 4. The nucleic acid sequence of the VZV ORF47 kinase gene and the amino acid sequence corresponding thereto are shown in SEQ ID No. 5 or 6. Besides these nucleic acid sequences the viral kinase may also be coded by a sequence within the scope of the degeneration of the genetic code, i.e. a sequence coding for a protein having the same amino acid sequence, or by a nucleic acid sequence hybridizing thereof under stringent conditions. According to Sambrook et al., Molecular Cloning. A Laboratory Manual, Cold Spring Harbor, Laboratory Press (1989) 1.101-1.104, stringent hybridization conditions are defined such that after washing for one hour with 1×SSC and 0.1% SDS at 55° C., preferably at 62° C., and particularly preferred at 68° C., particularly for 1 hour with 0.2×SSC and 0.1% SDS at 55° C., preferably at 62° C. and particularly preferred at 68° C., still a positive hybridization signal is observed.

For the method of the invention a kinase substrate is selected which is capable of being phosphorylated by the chosen kinase and wherein the phosphorylated substrate (either the substrate itself or a metabolite thereof) is deleterious, e.g. cytotoxic for the chosen target cell. For UL97 kinase from HCMV, and other viral kinases such as HSV UL13, VZV ORF47, HHV-6 ORF69, EBV BGLF4, HHV-8 ORF36 or homologous kinases, ganciclovir, aciclovir, famiciclovir, and other derivatives thereof are suitable substrates. It is evident, however, that the method of the present invention is widely applicable for a great variety of different kinases.

The determining step (d) of the method of the present invention may be qualitative. Preferably, however, the determining step comprises a quantitative measurement of the deleterious, e.g. cytotoxic effect mediated by the phosphorylated substrate. This quantitative measurement may be carried out by determining signals in the supernatant of the cultured cells, e.g. colour conversion of a phenol red-supplemented medium, and/or in the target cell, e.g. lactate dehydrogenase (LDH) activity in cell lysates as measured by an established cytotoxicity kit. The method of the invention is capable of being automated. Thus it may be carried out as a high-throughput screening of candidate compounds for kinase-specific therapeutical drugs. The methods for said quantitative measurement of the deleterious effect as carried out in the determining step (d) are not limited to the above-mentioned specific methods. Any suitable method known to a person skilled in the art can be used in order to obtain the desired results.

A further advantage of the present invention resides in the fact that only such test compounds are identified as kinase inhibitors which do not exhibit inadequately high cytotoxic side effects at the test concentration. If a test compound is capable of inhibiting the kinase, but additionally has a cytotoxic activity, no rescue from cell death would be observed. Thus, the method of the invention preferably comprises the additional step (e) distinguishing between (i) noncytotoxic test compounds having kinase inhibiting properties and (ii) test compounds having kinase inhibiting properties but additional cytotoxic side effects. Furthermore, the effect of a given test compound may be determined at several different concentrations of the test compound in order to obtain a more accurate information of the kinase inhibiting properties and possible unwanted cytotoxic side effects. Moreover, the effect of a test compound may be determined on a control cell, e.g. a target cell which does not contain the nucleic acid coding for the kinase to be tested or alternatively a target cell comprising a nucleic acid encoding an inactive variant of the kinase to be tested. In a particular preferred embodiment a determination of a given test compound is carried out at several different concentrations in target cells (expressing an active kinase) and control cells (not expressing an active kinase). In this manner, the concentration dependency and the target specificity of the inhibitory effect of the test compound and the concentration dependency of a possible cytotoxic effect may be determined together.

Thus, a major advantage of the in-cell-activity assay, particularly the UL97 assay, is that cytotoxicity can easily be taken as an indicator of kinase activity and that kinase inhibition leads to an increased survival of the cultured cells. By this means, an inherent cytotoxic effect of a putative inhibitory compound is immediately recognized.

For example, a nonspecific signal, in terms of an inherent cytotoxicity, was produced by the putative inhibitor staurosporine (STP), as detected with both mutants (FIG. 6: C and D). Thus, by comparing the panels, the nonspecific cytotoxicity of STP seen with mutant and wild-type versions of pUL97 (note that mutant K355M is catalytically inactive) could be clearly distinguished from the specific pUL97-inhibiting effect of NGIC-I only detected with wild-type pUL97.

An important goal of the present invention is to characterize chemical compounds with regard to their inhibitory properties towards specific kinases, preferably in combination with the presence and/or the strength of possible cytotoxic side effects. Further, the present invention allows determining the effect of the presence or absence of co-transfected nucleic acids, particularly co-transfected genes in the target cell. By using virus infected target cells the capability of infectious or defective viruses interfering with or enhancing the kinase activity can be determined.

Still another aspect of the present invention is the reagent kit for the identification of kinase inhibitors comprising a cell containing a nucleic acid encoding a kinase and a substrate capable of being phosphorylated by said kinase and wherein said phosphorylated substrate is deleterious for said target cell. The cell and the substrate should be kept in separate containers or compartments until the actual assay for the identification of kinase inhibitors is carried out. The reagent kit is preferably used in a method as described above.

Furthermore, a preferred protocol for the rapid and stable selection of transfected cell clones expressing the pUL97 kinase in an enzymatically active form is disclosed (High-throughput screening systems). We made efforts to select 293 cell clones transfected with pcDNA-UL97 which should stably express high amounts of the pUL97 kinase. Initially, however, it was difficult to maintain positive cell clones along higher passage numbers. Although several of the clones continued to express pUL97, as detectable by Western blot analysis, different tests for kinase activity were repeatedly negative. It seemed suggestive that the selection of inactive, spontaneously derived mutants of pUL97 kinase was favoured under these conditions (data not shown).

In order to improve the test performance, we developed a double selection protocol for those clones exclusively expressing a kinase, e.g. pUL97 kinase in an active state (FIG. 7: A). After transfection of the target cell with a vector comprising the kinase gene and a selection marker, e.g. geneticin and subcultivation of selection marker-resistant cells, individual clones were subjected in parallel to selection for either selection marker resistance alone or for resistance in addition to the ability to convert the substrate, e.g. GCV. Those clones identified to possess kinase activity were multiplied and used for screening experiments. As an example, cell clone 293-UL97 F10, directly incubated with NGIC-I during cultivation, indicated a clear sensitivity to the compound (FIG. 7: B): 50 nM of NGIC-I reduced the pUL97 kinase activity significantly. As a control, the vector-transfected cells (293-mock) did not produce signals of kinase activity (FIG. 7: C). The long-term passaging of different clones of UL97-expressing cells eventually led to a decrease in expression efficiencies, however, we could demonstrate for two independent cell clones that pUL97 remained clearly detectable for defined passage numbers and periods of analysis (FIG. 7: D). Within a range of two months, expression and activity of pUL97 was sufficiently high for kinase analysis and no changes in the growth behaviour of the cultures were observed. Moreover, the inhibitor NGIC-I was regularly used as a control in individual screening experiments, showing identical properties of inhibition throughout the period of testing (data not shown). Thus, the results obtained with 293 cell clones stably expressing pUL97 provide a confirmation of our data on pUL97-specific inhibition and deliver the basis for a screening system in larger scales.

Furthermore, the assay has been automatized and optimized to increase the screening throughput significantly. First, a stable 293 cell line stably expressing UL97 has been created to increase the reproducibility of the screening assay. Additionally, different cell quantitation methods were established to ensure a faster and easier read-out. A detailed description of the optimized screening-protocol is given under 2.

Further, the invention should be explained by the following figures and examples.

Figure Legends

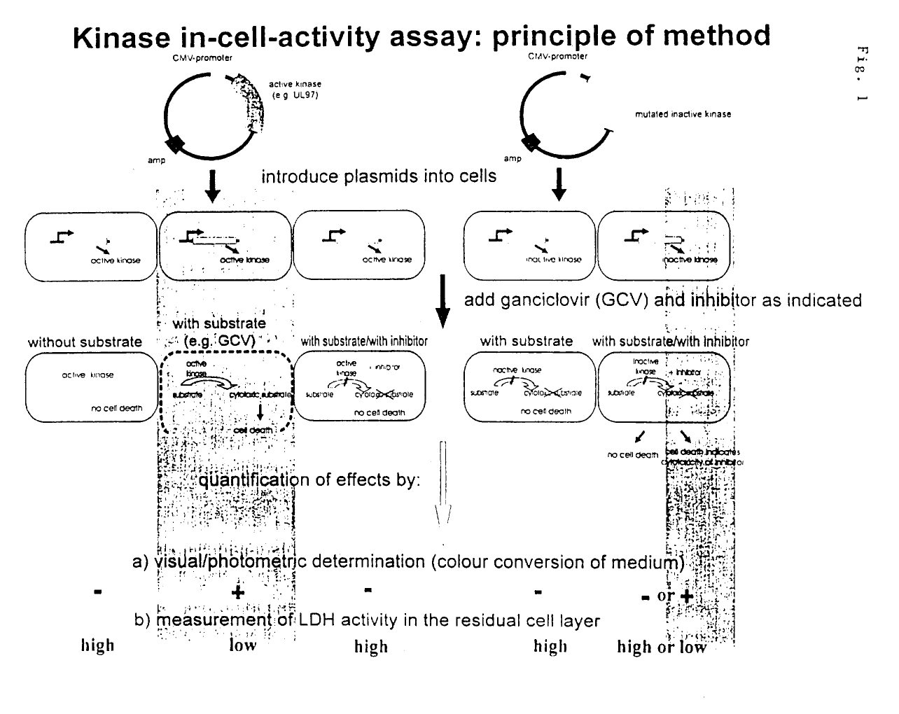

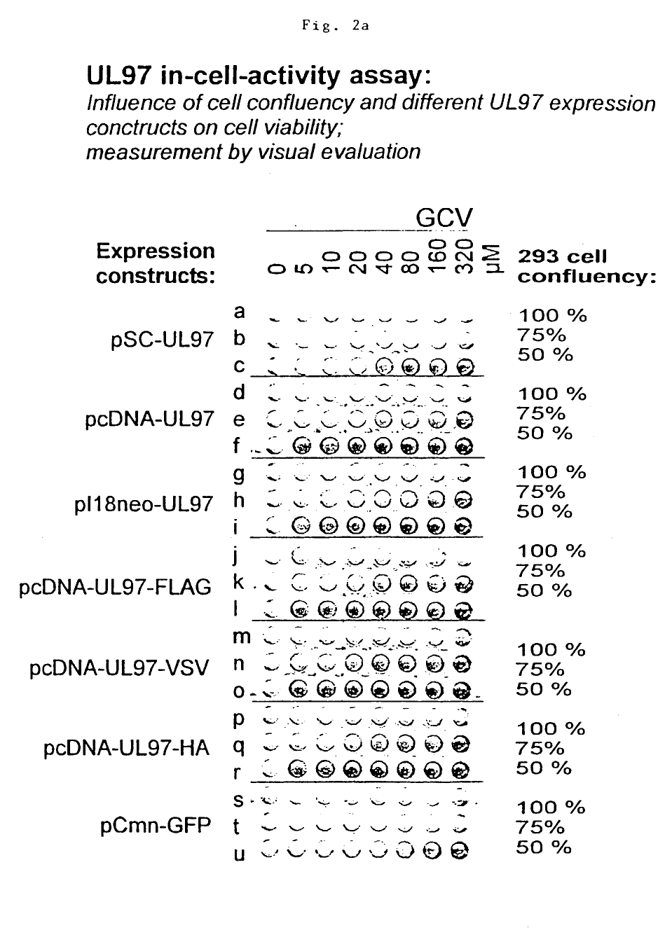

FIG. 1:

Schematic diagram illustrating the principle of the kinase in-cell-activity assay.

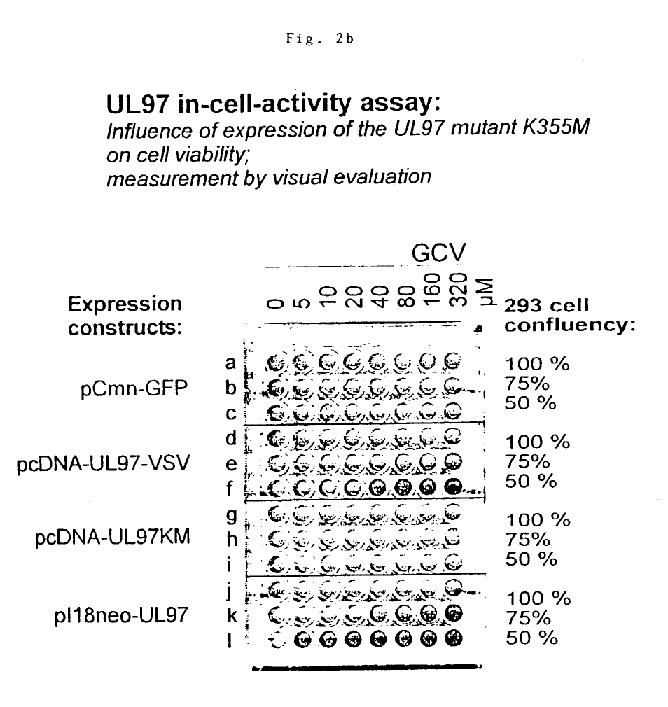

FIG. 2:

Establishment of the UL97 in-cell-activity assay.

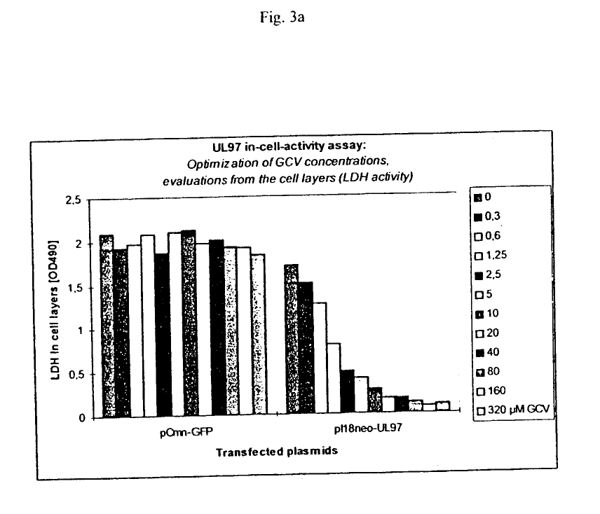

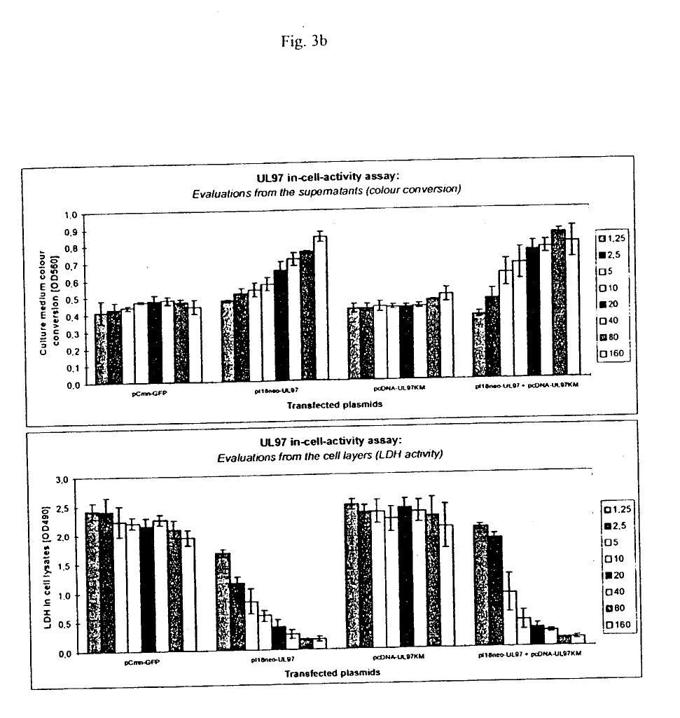

FIG. 3:

Optimization of GCV concentrations.

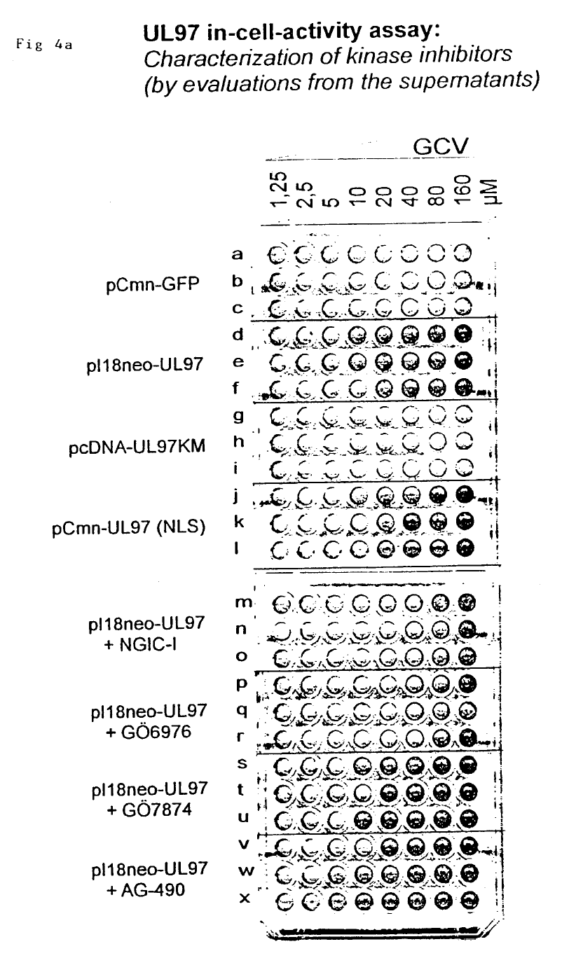

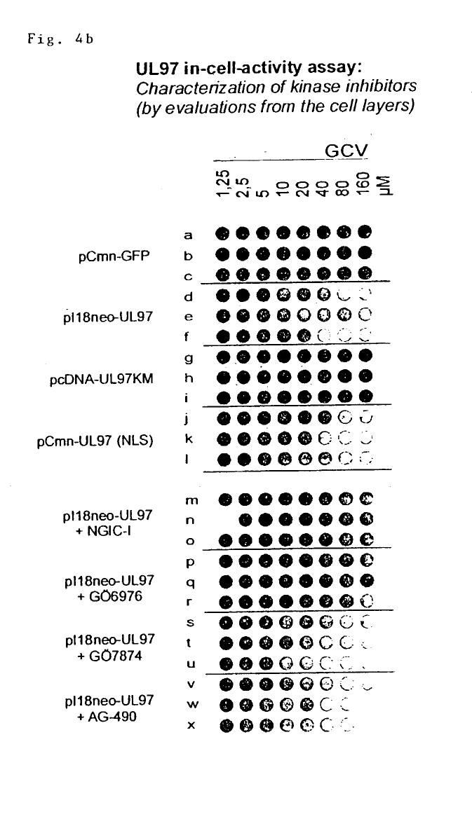

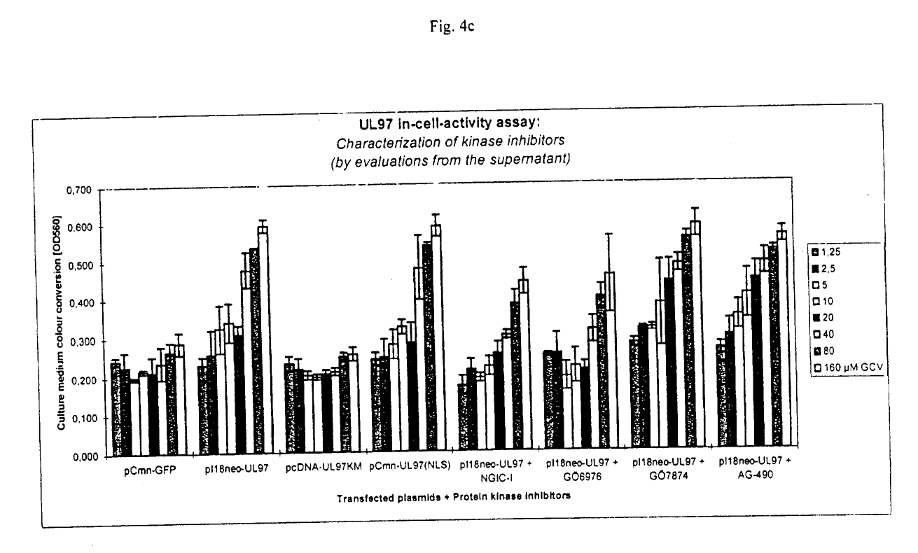

FIG. 4:

Characterization of kinase inhibitors by the use of the UL97 in-cell-activity assay.

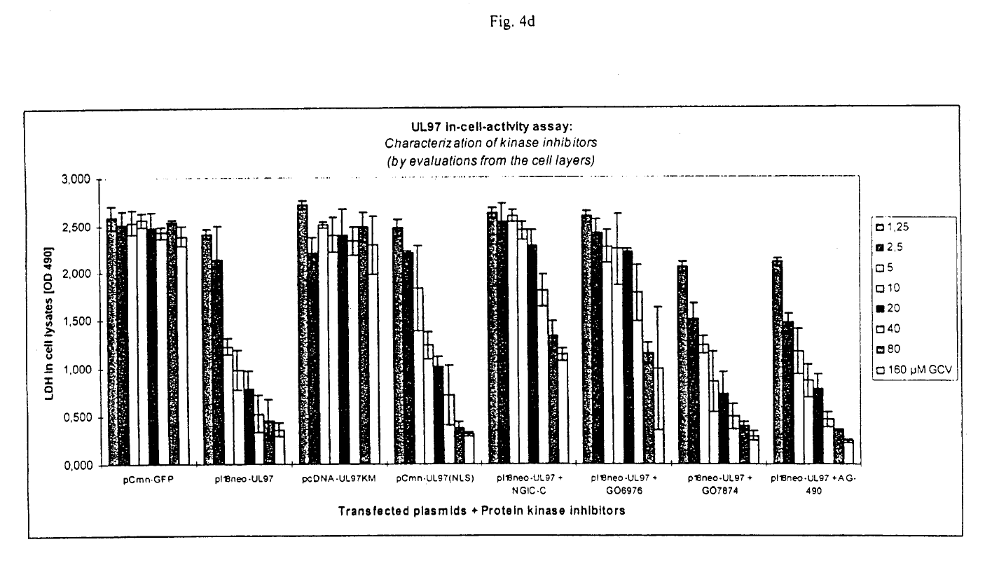

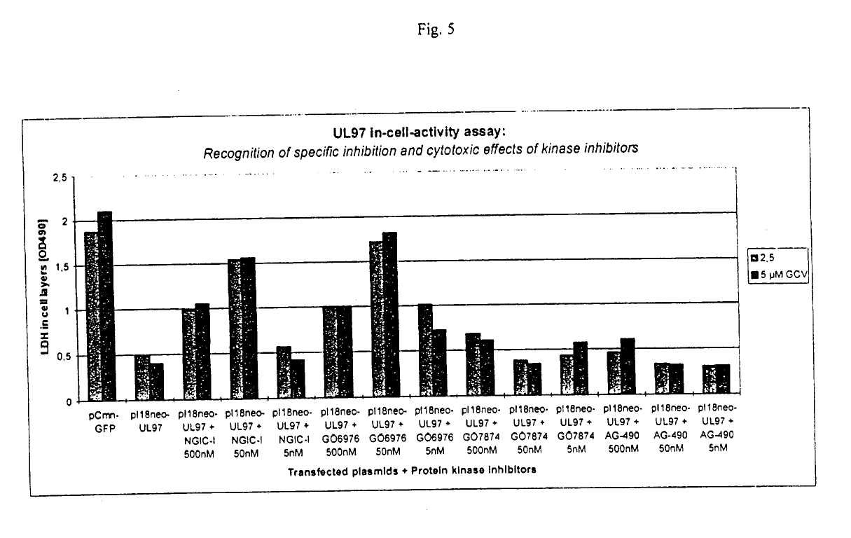

FIG. 5:

Recognition of specific inhibition of UL97 activity and of cytotoxic side-effects induced by the kinase inhibitors.

FIG. 6:

Activity of the wild-type and mutant versions of the pUL97 kinase

FIG. 7:

Double selection of cell clones stably expressing pUL97 in an active form

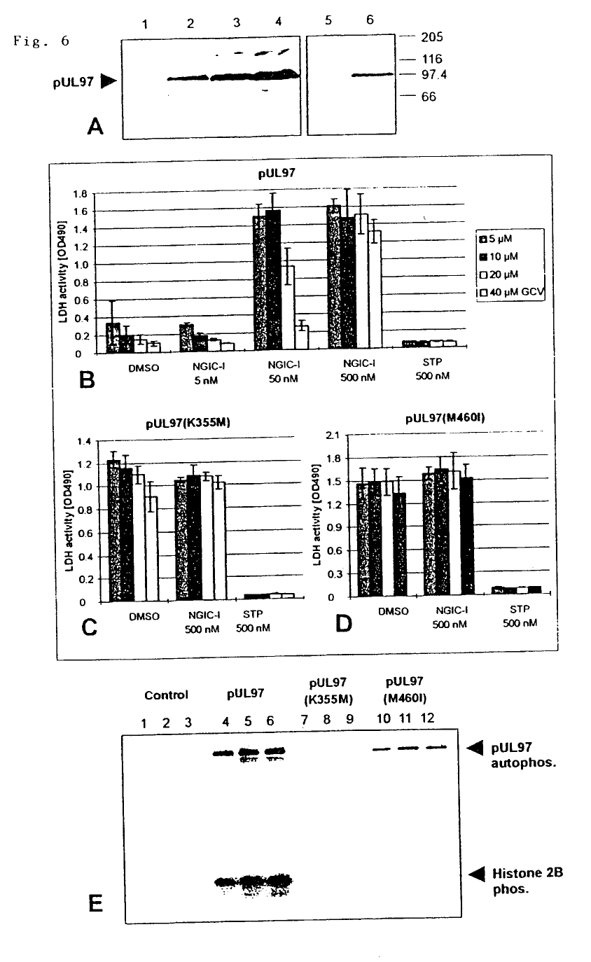

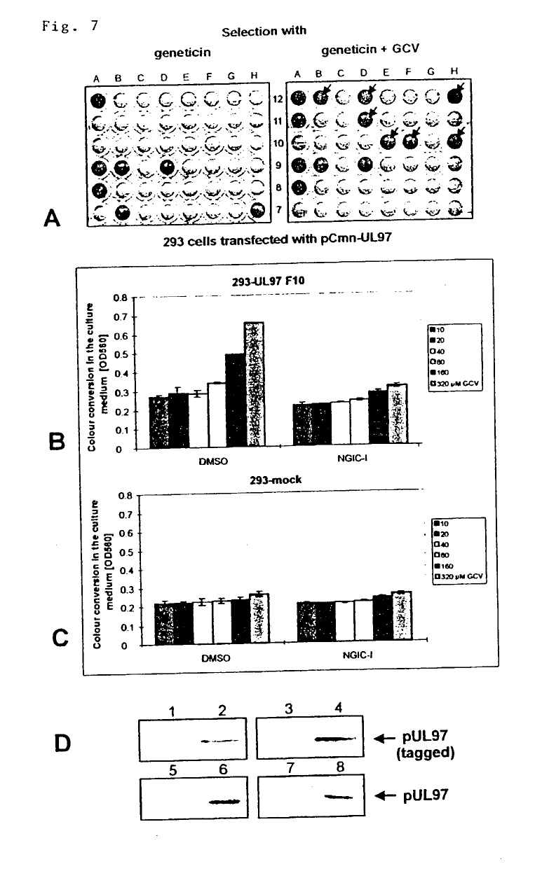

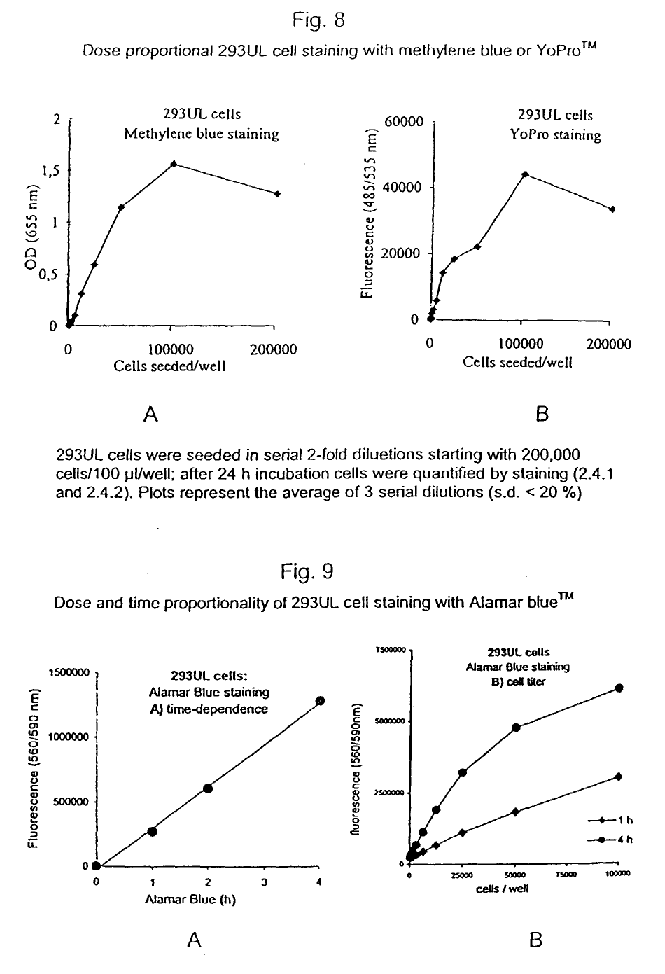

FIG. 8:

Dose proportional 293UL cell staining with methylene blue or Yopro™

FIG. 9:

Dose and time proportionality of 293UL cell staining with Alamar blue™

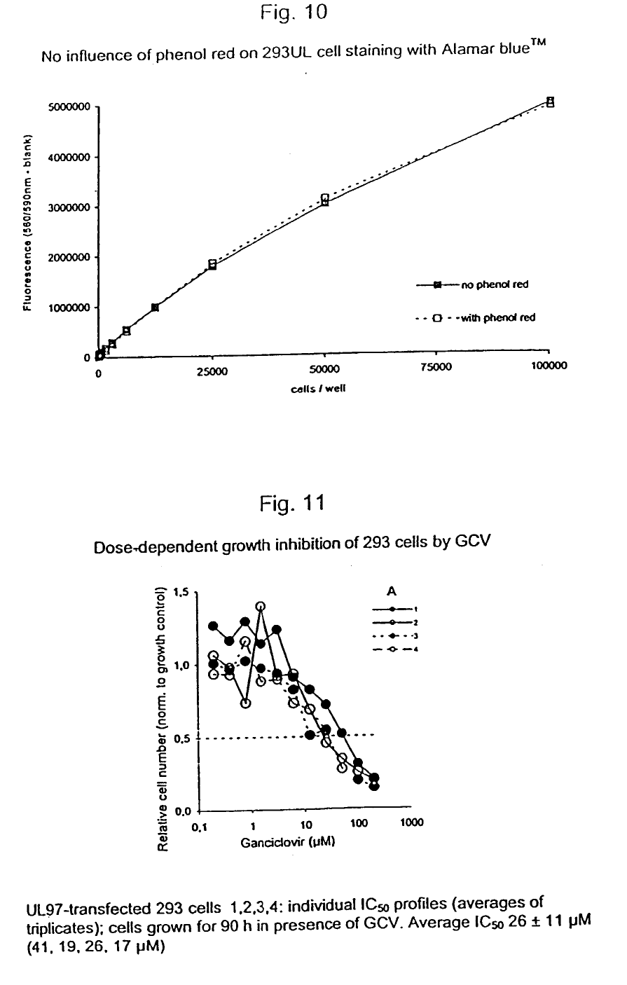

FIG. 10:

No influence of phenol red on 293UL cell staining with Alamar blue™

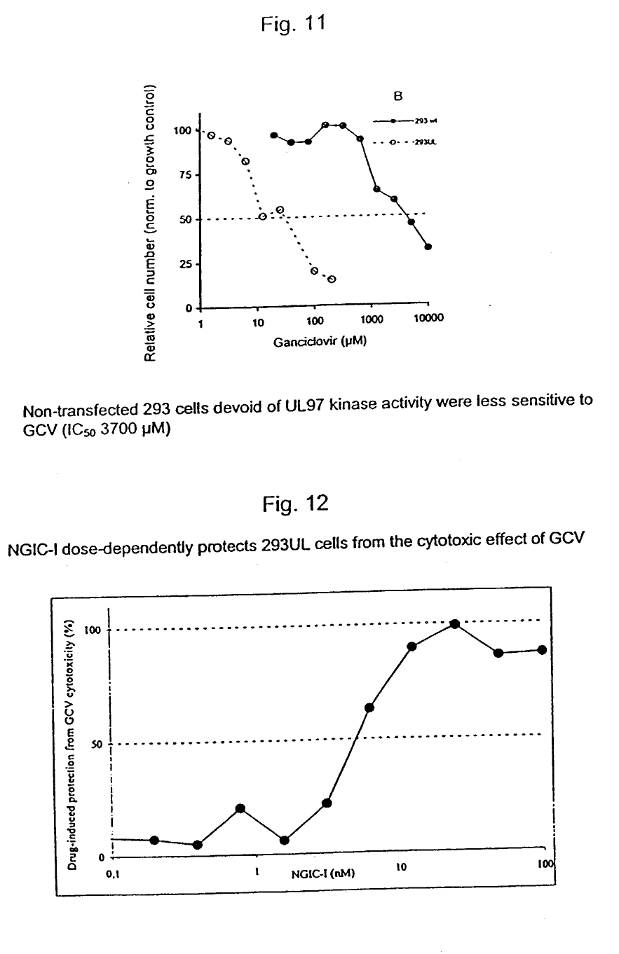

FIG. 11:

Dose-dependent growth inhibition of 293 cells by GCV

FIG. 12:

NGIC-I dose-dependently protects 293UL cells from the cytotoxic effect of GCV

### EXAMPLES

### 1. Plasmid Constructs and Sequences

SC-UL97

The ORF UL97 of the HCMV genome AD169 (Genebank Accession Number X17403, nucleotides 140,484-142,607) was amplified by PCR using primers 5-UL97-BgIII (TAGT AGATCT ATGTCCTCCGCACTTCGGTCT) and 3-UL97-SaII (TAGT GTCGAC TTACTCGGGGAACAGTTGGCG. The PCR product was digested with BgIII and SaII and inserted into vector pSuperCatch (Georgiev et al. 1996, Gene 168:165-167) via cloning sites BamHI and SaII.

pcDNA-UL97.

The ORF UL97 of the HCMV genome AD169 was amplified by PCR using primers 5-UL97-BgIII (TAGT AGATCT ATGTCCTCCGCACTTCGGTCT) and 3-UL97-SaII (TAGT GTCGAC TTACTCGGGGAACAGTTGGCG). The PCR product was digested with BgIII and SaII and inserted into vector pcDNA3 (Invitrogen) via cloning sites BamHI and XhoI.

pl18neo-UL97

The ORF UL97 of the HCMV genome AD169 was amplified by PCR using primers 5-UL97-BgIII (TAGT AGATCT ATGTCCTCCGCACTTCGGTCT) and 3-UL97-SaII (TAGT GTCGAC TTACTCGGGGAACAGTTGGCG). The PCR product was digested with BgIII and SaII and inserted into vector pl18neo (Marschall et al. 1999, Virology 253:208-218) via cloning sites BamHI and SaII.

pCmn-GFP:

pCmn is an internal designation for the pCMV/myc/nuc-vector purchased from Invitrogen (Invitrogen 1999 Product Catalog, p. 103; Fischer-Fantuzzi, L. and Vesco, C. (1988) Mol. Cell. Biol. 8: p. 5495-5503). The pCMV/myc/nuc-vector carrying a GFP expression motive (pCmn-GFP) instead of the UL97 insert was used as a positive control for pCmn-UL97.

pCmn-UL97:

The ORF UL97 of the HCMV genome AD169 (Genbank accession number X17403, nucleotides 140484-142607) was amplified by PCR using primers 5-UL97-NcoI (CATGCCATGGGCATGTCCTCCGCACTT) and 3-UL97-XhoI (CCGCTCGAGCTCGGGGAACAGTTG). The PCR product was digested with NcoI and XhoI and inserted into vector pCMV/myc/nuc (Invitrogen) via cloning sites NcoI and XhoI.

pcDNA3-UL97-FLAG:

The ORF UL97 of the HCMV genome AD169 (Genbank accession number X17403, nucleotides 140484-142607) was amplified by PCR using primers 5-UL97-EcoRI (CCCGAATTCATGTCCTCCGCACTTCGG) and 3-UL97-FLAG-XhoI (CCGCTCGAGTTACTTGTCGTCATCGTCTTTGTAGTCCTC GGGGAACAGTTG). The PCR product was digested with EcoRI and XhoI and inserted into vector pcDNA3 purchased from Invitrogen (Invitrogen 1994 Product Catalog, p. 51; Akrigg, A. et al. (1985) Virus Research 2: 107-121; Boshart, M. et al. (1985) Cell 41: 521-530) via cloning sites EcoRI and XhoI.

pcDNA3-UL97-VSV:

The ORF UL97 of the HCMV genome AD169 (Genbank accession number X 17403, nucleotides 140484-142607) was amplified by PCR using primers 5-UL97-EcoRI (CCCGAATTCATGTCCTCCGCACTTCGG) and 3-UL97-VSV-XhoI (CCGCTCGAGTTACTTGCCCAGCCGGTTCATCTCGATGTCGGTG TACTCGGGGAACAGTTG). The PCR product was digested with EcoRI and XhoI and inserted into vector pcDNA3 purchased from Invitrogen (see above) via cloning sites EcoRI and XhoI.

pcDNA3-UL97-HA:

The ORF UL97 of the HCMV genome AD169 (Genbank accession number X17403, nucleotides 140484-142607) was amplified by PCR using primers 5-UL97-EcoRI (CCCGAATTCATGTCCTCCGCACTTCGG) and 3-UL97-HA-XhoI (CCGCTCGAGTTAAGCGTAATCTGGAACATCGTATGGGTACT CGGGGAACAGTTG). The PCR product was digested with EcoRI and XhoI and inserted into vector pcDNA3 purchased from Invitrogen (see above) via cloning sites EcoRI and XhoI.

pcDNA3-UL97-K355M:

The ORF UL97 of the HCMV genome AD169 (Genbank accession number X17403, nucleotides 140484-142607) was amplified by PCR using primers 5-UL97-EcoRI (CCCGAATTCATGTCCTCCGCACTTCGG) and 3-UL97-XhoI (CCGCTCGAGTTACTCGGGGAACAGTTG). The PCR product was digested with EcoRI and XhoI and inserted into vector pcDNA3 purchased from Invitrogen (see above) via cloning sites EcoRI and XhoI. This construct was used to perform site directed mutagenesis (Kunkel et al., PNAS 82, (1985) 488-492) to substitute the Lysin at position 355 by Methionin. The following mutagenesis-primer was used: 5′-CTTACGCGCCACCATGACCACGCGATA-3′.

### 2. UL97 In-Cell-Activity Assay

2.1. Assay Development

The following protocol describes the assay conditions during the assay development phase:

Day 1.

Human embryonic kidney cells 293 (ATCC CRL-1573), as cultivated with DMEM medium containing 5% fetal calf serum (FCS), were harvested by trypsinization, rinsed with PBS and seeded in 96-well plates at a cell number of 20,000 per well.

Day 2.

Transfection (Lipofectamin Plus reagents, GibcoBRL) was performed at a cell layer confluency of 50-75%. For this, identical transfection conditions were chosen for 24 wells of the 96-well plate to obtain determinations over a 8-well line in triplicate. One transfection set-up for 24 wells was composed as follows:

Component A—2.5-10 μg plasmid DNA (for the expression of UL97, and optionally other genes), 300 μl DMEM-0% FCS, 25 μl Plus reagent.

Component B—12.5 μl Lipofectamin reagent, 300 μl DMEM-0% FCS. Both components were incubated for 15 min at room temperature. Then, components A and B were combined, mixed thoroughly and again incubated for 15 min at room temperature.

Meanwhile, culture media of the 96-well plates were removed by the use of a multichannel pipette and a 50-μl volume of fresh DMEM-0% FCS was given in each well. Then, 25 μl of each transfection set-up was added per well. Plates were incubated for 5 h at 37° C. in a 5% CO₂ atmosphere. Subsequent to this incubation, a 125-μl volume of DMEM-10% FCS was added per well and incubated over night at 37° C. in a 5% CO₂ atmosphere.

Day 3.

Transfection media were removed from the cells by the use of a multichannel pipette. Ganciclovir (GCV) was diluted in DMEM-5% FCS (in that a gradient of appropriate GCV concentrations was generated) and added in a volume of 100 μl per well. Kinase inhibitors were diluted in DMEM-5% FCS added in a volume of 100 μl per well, immediately after the addition of GCV. The plates were incubated at 37° C. in a 5% CO₂ atmosphere.

Day 8.

Visual and photometric determinations of the colour conversion of the phenol red-supplemented culture medium was performed by the use of a computer scanner and an ELISA reader (OD 560 nm).

Measurement of the lactate dehydrogenase (LDH) acitivity in the residual cell layer was performed with the CytoTox 96 Non-Radioactive Cytotoxicity (Promega). For this, culture media were removed, cells were rinsed with PBS and lysed in a 1× concentration of the kit lysis buffer (100 μl per well). After an incubation for 45 min at 37° C., the cell debris was removed by centrifugation and 5 μl of each lysate was diluted in a total of 50 μl PBS for the LDH measurement. 50 μl of substrate mix was added to each well and incubated for 30 min at room temperature in the dark. Thereafter, 50 μl of stop buffer was added and the colour reaction was quantitated by the use of an ELISA reader (OD 490 nm).

2.2. Screening:

The following protocol describes the assay upon optimization and standardization:

2.2.1. Generation and Isolation of UL97-Expressing 293 Cell Clones

293 cells transfected with pUL97 expression construct (pcDNA3-UL97; see above under 2.1.) were grown for 14 d in presence of geneticin (750 μg/ml). Individual clones were isolated (minimal dilution), re-tested for geneticin resistance and for sensitivity to 100 μM GCV. Clones identified as geneticin resistant and GCV-sensitive were expanded and stored frozen in aliquots for further experiments, after having verified by Western blot their capacity to express UL97 protein. As primary antibodies, we used serum of rabbits immunized with the UL97 peptide 1-16 (MSSALRSRARSASLGT), and for detection a peroxidase-labelled anti-rabbit serum; the readout was achieved with an enhanced chemoluminescence kit (Amersham).

2.2.2. Culture Conditions for UL97-Transfected 293 Cells

293UL cells were routinely grown in DMEM supplemented with 10% FCS, glutamine (1%), pyruvate (1%), geneticin (final 0.5 mg G418/ml culture), and penicillin/streptomycin (1%). Medium was changed every 3-4 d, and cells were subcultured before reaching confluency by trypsin/EDTA treatment (exposure of adherent cells to 5 mg/ml trypsin and 2 mg/ml EDTA, dissolved in sterile 0.85% NaCl) and re-seeding in at least 5-fold dilution. Cells were maximally 10 times subcultured before a new frozen batch was used.

2.2.3. Initial Layout

For routine antiproliferative assays, 293 UL cells were suspended at 13.8×10³ cells/ml in complete medium, and 145 μl/well (2000 cells) of this suspension were seeded in 96-well flat-bottom plates (Nunclon #167008). After attachment of the cells for 24 h, drugs were added: All wells received 5 μl GCV (4 mM in complete medium, final concentration 100 μM), and each 3 wells received 50 μl compound (40 μM in complete medium+0.4% DMSO). A control series received 50 μl 40 μM compound only in the absence of GCV. Another control plate, which had received cells or medium only, was glutaraldehyde-fixed or frozen (see below) at the time of drug addition and stained for quantitation of cell density.

In some experiments, 100 μM test compound was used in presence of 1% DMSO; in those experiments all control cultures also contained 1% DMSO.

On every plate, control wells were included (row H, final concentrations):

| | |
|---|---|
| well 1-3: | growth control (DMSO, no drugs) |
| well 4-6: | 0.1% DMSO + GCV series (400, 100, 25 μM) |
| well 7-9: | 0.1% DMSO + 100 μM GCV + 3, 10, 30 nM NGIC-I |
| well 10-12: | sterile control (0.1% DMSO, no cells) |

Cells were further incubated for 72 h and then analyzed for proliferation (staining with methylene blue or fluorescent dyes, see section 2.4).

The reader data were exported into Excel™ and calculated.

2.2.4. Calculation of UL97 Inhibition

| | | |
|---|---|---|
| 100% cell growth | 3 wells cells without | OD(growth control) |
| | added drug | |
| background | 3 wells without cells | OD(sterile control) |
| 100% UL97 activity | 3 wells with cells + GCV only | OD(GCV) |
| drug + GCV treated | 3 wells with cells + GCV + | OD(drug + GCV) |
| | drug | |

$Effect of drug X \text{:} 100 \cdot \frac{OD ( drug + GCV ) - OD ( GCV )}{OD ( growth control ) - OD ( GCV )}$

Prior to calculation of this ratio, background (3 cell-free wells) and start OD had been subtracted from the averages of all triplicates.

Examples: Perfect UL97 inhibitor: (drug+GCV)-treated cells grow like growth control

$$OD ( drug + GCV ) = OD ( growth control )$$

$$100 \cdot \frac{OD ( drug + GCV ) - OD ( GCV )}{OD ( growth control ) - OD ( GCV )} = 100$$

Drug inactive against UL97: (drug+GCV)-treated cells grow like GCV treated cells

$$OD ( drug + GCV ) = OD ( GCV )$$

$$100 \cdot \frac{OD ( drug + GCV ) - OD ( GCV )}{OD ( growth control ) - OD ( GCV )} = 0$$

2.2.5. Calculation of Cytotoxicity

| | |
|---|---|
| 100% cell growth | 3 wells without added drug OD(growth control) |
| background | 3 wells without cells OD(sterile control) |
| Drug cytotoxicity | 3 wells with drug only OD(drug) |
| | $100 \cdot \frac{OD ( drug )}{OD ( growth control )}$ |

Non-toxic drug: >>50 (close to 100; drug-treated culture grows as well as drug-free control)
Toxic drug: <<50 (drug-treated culture contains much less cells than drug-free control)

2.2.6 Screening

The experimental layout was maintained as described in the previous section with the exception, that the compound distribution was changed for adaptation to a Packard Multiprobe pipetting station. Column 12 of the multiwell plate was kept for controls:

| | |
|---|---|
| A12 | growth control (cells, no drug) |
| B12, C12, D12 | GCV (25, 100, 400 μM) |
| E12, F12, G12 | 100 μM GCV + NGIC-I (30, 10, 3 nM) |
| H12 | sterile control (no cells, no drugs) |

Triplicate plates were run for toxicity (compounds alone) and for UL97 inhibition (compounds in presence of 100 μM GCV).

For screening, cells were quantitated with the Alamar blue™ assay (see below)

2.2.7. Cell Quantitation

2.2.7.1. Methylene Blue

add 40 μl 25% glutaraldehyde to 200 μl cell culture, keep 10 min at ambient temperature.

remove liquid, wash once with water

add 50 μl methylene blue (0.05% in water, filtered), keep 10 min at ambient temperature

remove dye, wash three-fold with water

add 200 μl 3% HCl, incubate 1 h on shaker (100 rpm)

Read OD_{655 nm} in a BioRad reader (Benchmark)

2.2.7.2. YoPro™

add 10 volume-% of 20 μM YoPro™ in 10×lysis buffer (20 mM EDTA, 20 mM EGTA, 1% NP-40)

shake for 30 min at 100 rpm

Read fluorescence on a Tecan Ultra or a Victor II/5 reader (Wallac) at 485 nm excitation and 525 -535 nm emission.

FIG. 8 Dose proportional 293UL cell staining with methylene blue or YoPro™

293UL cells were seeded in serial 2-fold dilutions starting with 200,000 cells/100 μl/well; after 24 h incubation cells were quantitated by staining (2.4.1 and 2.4.2). Plots represent the average of 3 serial dilutions (s.d.<20%)

2.2.7.3 Alamar Blue™

The dye Alamar blue™ (Serotec/Biozol; BUF012) changes its fluorescent properties upon reduction. It is water soluble and permeates cell membranes making it versatile for cell quantitation based on dye reduction by cellular metabolic enzymes.

Alamar blue™ staining of cells grown in the presence or absence of phenol red yielded similar fluorescence (FIG. 3); only at cell titers below 5000/well (below a fluorescence intensity 2- to 3-fold above reagent background) phenol red caused a significant reduction of fluorescence.

Add 10% dye stock to growing cell culture

Incubate cells for 3-4 h (conversion of the dye from the oxidized to the reduced form enables the quantitative detection of up to 30,000 293UL cells/well in a 96-well plate).

Record with a Victor II/5, excitation 560 nm, emission 590 nm). Shorter and longer incubation periods and multiple recordings of dye conversion are possible.

FIG. 9 Dose and time proportionality of 293UL cell staining with Alamar blue™

A) 293UL cells were seeded at a density of 2000 cells/200 μl/well, grown for 96 h, and then 20 μl Alamar blue™ added. After 1, 2, and 4 h incubation, fluorescence was recorded (2.4.2; excitation 560 nm, emission 590 nm). Averages of triplicate determinations after subtraction of reagent blank (184,000-203,000) are plotted (s.d.<10%).

B) 293UL cells were seeded in serial 2-fold dilutions starting with 100,000 cells/100 μl/well, and after 24 h incubation cells were quantitated by staining as described (2.4.2). Plots represent the average of 7 serial dilutions (s.d.<5%), Alamar blue™ incubation was recorded after 1 and 4 h (FIG. 9).

FIG. 10 No influence of phenol red on 293UL cell staining with Alamar blue™

Each 4 rows with serial two-fold dilutions of 293UL cells starting with 100,000 cells/well were seeded in a 96-well plate in complete medium with or without phenol red, grown for 24 h, and then stained with Alamar blue™. Averages are plotted after subtraction of reagent blanks (s.d.<10%).

### 3. Results

3.1 General Description (FIG. 1)

The principle of the kinase in-cell-activity assay is shown in FIG. 1. Plasmids encoding either an intact kinase (e.g. UL97 kinase encoded by human cytomegalovirus) or a kinase inactive mutant are introduced into cells (e.g. by transfection). Either an intact kinase or an inactive protein (serving as a control for non-specific effects) are expressed within the cells. Substrate (e.g. ganciclovir) is then added in an appropriate concentration to transfected cells (as indicated in the diagram). Moreover, potential inhibitors of kinase activity are also added as indicated. In the presence of an active kinase, the substrate (e.g. ganciclovir=GCV) is converted into a cytotoxic drug. For example, in case of UL97, GCV is converted to its monophosphorylated form (GCV-P) which is further converted by cellular enzymes to the triphosphorylated form (GCV-PPP). GCV-PPP exerts toxic effects ultimately resulting in cell death. In the presence of a kinase inhibitor, conversion of the substrate to the cytotoxic form is blocked. Thus, cell death is prevented. As a control, cells expressing an inactive kinase mutant are incubated together with the substrate and the kinase inhibitor. If cell death can be observed with the inactive kinase this indicates cytotoxicity of the inhibitory substance. As a measurement for cell death, either the colour conversion of the medium (containing phenol red as a pH indicator) can the quantified photometrically, or the LDH activity within the residual cell layer can be determined (resulting in low activities when extensive cell death has occurred). Other methods of quantification are also possible (e.g. measurement of cell proliferation).

3.2 Establishment of the UL97 In-Cell-Activity Assay (FIG. 2)

293 cells were seeded on 96-well plates at different cell numbers and cultivated until reaching a confluency of 100%, 75% or 50%. Then the cells were transfected with the indicated expression constructs and incubated with GCV (concentrations ranging from 5 μM to 320 μM) or without GCV. Five days after the addition of GCV, a qualitative/semi-quantitative determination of the GCV-mediated cytotoxic effect in the presence of active UL97 kinase was performed by computer scanning of the plates and by visual evaluation.

FIG. 2 shows that the GCV-mediated effect was indicated by a colour conversion of the phenol red-supplemented culture medium from yellow to red (compare the negative vector control pCmn-GFP). Best signals were obtained at a cell confluency of 50%. All constructs expressing UL97 (including tagged versions) were positive, i.e. cytopathic effects could be observed as indicated by the red colour of the culture medium, while constructs expressing the inactive UL97 mutant K355M were negative.

3.3 Optimization of GCV Concentration (FIG. 3)

293 cells were seeded on 96-well plates at a cell number of 20,000 per well and cultivated until reaching a confluency of 50%. Then the cells were transfected with the indicated plasmids and incubated with GCV [0.3 μM to 320 μM in (FIG. 3a); 1.25 μM to 160 μM in (FIG. 3b)] or without GCV. Five days after the addition of GCV, the read-out of signals was performed by the measurement of LDH activity in the residual cell layers. Additionally, a photometric quantification of the colour conversion of the culture medium was performed in (b). Determinations were made in duplicate for (a) and in triplicate for (b).

Note that gradual degrees of the GCV-mediated effect were measurable in the presence of active UL97 kinase over the complete range of GCV concentrations tested. Cotransfection of a construct expressing active kinase in a 1+1 ratio to a construct expressing the inactive kinase mutant, did not prevent the effect resulting from the coexpressed active UL97 kinase.

3.4 Characterization of Kinase Inhibitors (FIG. 4)

293 cells were seeded on 96-well plates at a cell number of 20,000 per well and cultivated until reaching a confluency of 50%. Then the cells were transfected with the indicated plasmids and incubated with GCV (1.25 μM to 160 μM). In addition to GCV, UL97-expressing cells were treated with 50 nM of one of four protein kinase inhibitors, i.e. NGIC-I (Kleinschroth et al., Bioorg. Med. Chem 3 (1993), 1959), GÖ6976 (Geschwendt et al., FEBS Lett. 392 (1996), 77), GÖ7874 (Kleinschroth et al., Bioorg. Med. Chem 3 (1995), 55) or AG-490 (Meydan et al., Nature 379 (1996), 645-648), respectively (Calbiochem).

Five days after addition of the substances, the read-out of signals was performed by the photometric quantification of the colour conversion of the culture medium and, for comparison, by the measurement of LDH activity in the residual cell layers. Qualitative/semi-quantitative illustrations are given by computer scanning figures in (a) for colour conversion, and in (b) for LDH activity. Quantitative values are presented as the results of photometric determinations in (c) for colour conversion, and in (d) for LDH activity. All data were produced in triplicate and standard deviations are indicated (as an exception, value n-1.25 only results from a duplicate calculation due to the contamination of one well).

The substances NGIC-I and GÖ6976 were identified as UL97-specific inhibitors, whereas GÖ7874 and AG-490 were not. Concerning the two modes of read-out, both measurements of colour conversion (newly developed) and LDH activity (standard test) were identical in specificity, providing reliable evidence for UL97 activity or inhibition; sensitivity of the LDH measurement was higher.

3.5 Determination of Specific Inhibition of UL97 Activity and Cytotoxic Side Effects (FIG. 5)

293 cells were seeded on 96-well plates at a cell number of 20,000 per well and cultivated until reaching a confluency of 50%. Then the cells were transfected with the plasmid pl18neo-UL97, expressing the active UL97 kinase, or vector pCmn-GFP as a control, and incubated with optimal concentrations of GCV (2.5 μM and 5 μM). In addition to GCV, UL97-expressing cells were treated with 5 nM, 50 nM or 500 nM of one of the four protein kinase inhibitors NGIC-I, GÖ6976, GÖ7874 and AG-490. Five days after the addition of the substances, LDH activity was determined from lysates of the residual cell layers.

The data of the UL97 activity without addition of substances compared to those including one of the substances clearly show a specific inhibition of UL97 activity by NGIC-I and GÖ6976, which was stronger at the 50-nM than at the 5-nM concentration. Importantly, at the highest concentration of 500 nM, inhibition of UL97 activity was not further enhanced (neither did it remain on an equal level) but was markedly decreased. This decrease in LDH activity indicates cytotoxicity of the inhibitory substance. For instance, the relative cytotoxicity (with respect to the vector control) was calculated as

4.5±1.0-fold for UL97 alone,

4.2±1.2-fold for UL97 plus 5 nM NGIC-I

1.3±0.1-fold for UL97 plus 50 nM NGIC-I and

2.0±0.1-fold for UL97 plus 500 nM NGIC-I.

Thus, the increased relative cytotoxicity at 500 nM, compared to 50 nM, quantitatively describes the cytotoxicity of NGIC-I at the higher concentration.

3.6 Activity of the Wild-Type and Mutant Versions of the pUL97 Kinase (FIG. 6)

(A) 293 cells were transfected with plasmids pcDNA-UL97 (wild-type; lane 2), pcDNA-UL97(K355M) (catalytically inactive mutant; lane 3), pcDNA-UL97(M4601) (GCV-resistant mutant; lane 4) or mock-transfected (pcDNA-3; lane 1), harvested 2 d posttransfection and analyzed by Western blotting. As a control, HFF infected with HCMV AD169 for 3 d (lane 6) or mock-infected (lane 5) were assayed. Blots were developed by the use of the pUL97-specific peptide antiserum, PepAs 1343. The pUL97-specific band is marked on the left and molecular weights are indicated on the right.

(B-D) 293 cells were transfected with the same plasmids as above, incubated with concentrations of GCV between 5 and 40 μM in the presence of the solvent DMSO or 5 nM to 500 nM of the inhibitors NGIC-I or STP, respectively. Five d posttransfection, LDH activity was determined from the residual cell layers using the cytotoxicity assay. The measurements were based on transfections in duplicate and all samples were used for double determinations of LDH activity (four values). Mean values and standard deviations are given.

(E) UL97 in vitro kinase assay was performed with precipitates from 293 cells transfected with the same plasmids as above. Autophosphorylation of pUL97 and the phosphorylation of exogenously added histone 2B is presented for the three versions of pUL97. All transfections were performed in triplicate and a control Western reblotting using UL97-specific antibodies (PepAs 1343) was performed to confirm that equal amounts of protein had been loaded (data not shown). Control, mock-transfected 293 cells.

3.7 Double Selection of Cell Clones Stably Expressing pUL97 in an Active Form (FIG. 7)

(A) 293 cells were transfected with plasmid pCmn-UL97 or pcDNA-3 (vector control) and selected for the formation of recombinant clones. After foci formation, individual clones were seeded in two plates in parallel and subjected either to a single selection with geneticin (left panel) or to a double selection with geneticin plus GCV (right panel). Those geneticin-resistant clones showing GCV sensitivity were identified by a colour conversion in the culture media (arrow-heads).

(B and C) Clones 293-UL97 F10 and 293-mock were cultivated in the presence of GCV (10 to 320 μM) and 50 nM of NGIC-I or the solvent DMSO, respectively. Five d postincubation, the colour conversion in the culture media was quantitated by a direct photometric determination. Double determinations and error bars are shown.

(D) In parallel settings, clones 293-UL97 F10 and 293-mock (described above; lanes 2-4) as well as 293-UL97^{Axx} and 293-mock^{Axx} (transfected with plasmid pLXSN-UL97 or vector pLXSN, respectively; lanes 5-8) were assayed on Western blots for the expression of pUL97. For clone 293-UL97 F10, samples were taken at passage numbers 4 (lane 2) and 16 (lane 4) posttransfection (lane 1, untransfected 293 cells; lane 3, clone 293-mock). For clone 293-UL97^{Axx}, samples were taken either immediately (lane 6) or 22 d (lane 8) posttransfection (lane 5, clone 293-mock^{Axx} immediately posttransfection; lane 7, clone 293-mock^{Axx} 22 d). Blots were developed by the use of UL97-specific antibodies (lanes 1-4, MAb-UL97; lanes 5-8, PepAs 1343).

The Protocol:

In a double selection protocol for UL97-expressing cell clones, 293 cells were transfected with pUL97 expression constructs or control plasmids encoding a geneticin-selectable marker and were selected for geneticin resistance (750 μg/ml). Individual clones were isolated and subjected in parallel to selection for either geneticin resistance alone (cell stock plate) or for resistance in addition to the ability to convert GCV at a concentration of 100 μM (activity test plate). Those clones identified to express active pUL97 kinase were multiplied from the cell stock plate and used for larger scales of screening compounds inhibiting pUL97 kinase activity.

3.8. Automatization, Optimization and Screening (FIGS. 11, 12)

3.8.1 Stable Transfection of UL97 Into 293 Cells

293 cells transfected with UL97 as described above stably expressed UL97 when grown in the presence of G418 (which was used as continuous selective pressure against loss of the plasmid). Expression was shown by presence of the UL97 protein in cell extracts, and by autophosphorylation activity of immunoprecipitated cell extracts.

UL97 expression was shown to persist over 10 cell transfers (2 months of growth) of UL97-transfected 293 cells: after this time cells still expressed the kinase-active UL97 protein.

3.8.2 Growth of 293UL Cells After Addition of GCV

Wild-type 293 cells are not affected in their proliferation by up to 100 μM GCV (FIG. 11B). The transformed cell line 293UL became sensitive to GCV with an IC₅₀ of 26±11 μM (s.d.) (FIG. 11A).

FIG. 11 Dose-dependent growth inhibition of 293 cells by GCV

A) UL97-transfected 293 cells 1,2,3,4: individual IC₅₀ profiles (averages of triplicates); cells grown for 90 h in presence of GCV. Average IC₅₀ 26±11 μM (41, 19, 26, 17 μM).

B) Non-transfected 293 cells devoid of UL97 kinase activity were less sensitive to GCV (IC₅₀ 3700 μM).

3.8.3 Drug-Induced Protection of 293UL Cells From GCV Cytotoxicity

293UL cells can be protected from GCV cytotoxicity by addition of a UL97 kinase inhibitor, e.g. NGIC-I (FIG. 12). This indolocarbazole shows potent in vitro inhibitory activity against UL97 kinase (IC₅₀ 1 nM; manuscript submitted to J. Gen. Virol.). When added to 293UL cells growing in the presence of 100 μM GCV (well above the growth IC₅₀), NGIC-I protects these cells from the cytotoxic effects of GCV with a 50% protection effect (PC₅₀) reached at 3-10 nM concentration.

FIG. 12 NGIC-I dose-dependently protects 293UL cells from the cytotoxic effect of GCV

293UL cells were seeded (2000 cells/well), and after 24 h drugs were added (100 μM GCV and serial two-fold dilutions of NGIC-I starting at 100 nM). After 3 d further incubation, cell mass was measured (2.4.1, methylene blue). GCV caused a 50% reduction of cell growth, and NGIC-I could completely overcome this (50% protection from GCV toxicity by 3.6-4.9 nM NGIC-I as determined with two different batches of cells). The graph represents the average of triplicate determinations; dilution series of NGIC-I tested in the absence of GCV caused no cytotoxicity (all wells contained 100-120% of the cell mass of drug-free controls).

3.8.4. Drug-Induced Cytotoxicity for 293UL Cells

Compounds exerting cytotoxic effects at the tested concentration cannot be detected as UL97 kinase inhibitors. Therefore 293UL cells are exposed to test compounds in the absence and presence of GCV. When drugs show cytotoxicity on 293UL cells, tests have to be repeated at lower drug concentration.

1. A method for the identification of kinase inhibitors comprising the steps:
(a) providing a target cell comprising a nucleic acid encoding a kinase,
(b) adding to the target cell a substrate wherein said substrate is capable of being phosphorylated by said kinase and wherein said phosphorylated substrate is deleterious for said target cell,
(c) adding to the target cell at least one test compound and
(d) determining, if said test compound is capable of at least partially inhibiting the deleterious effect of said phosphorylated substrate.

2. A method for the identification of kinase inhibitors comprising the steps:
(a) providing a target cell comprising a nucleic acid encoding a kinase,
(b) adding to the target cell a substrate wherein said substrate is capable of being phosphorylated by said kinase and wherein said phosphorylated substrate is deleterious for said target cell,
(c) adding to the target cell at least one test compound and
(d) determining, if said test compound is capable of at least partially inhibiting the phosphorylation of the substrate.

3. The method of claim 1 or 2 wherein said kinase is heterologous for said target cell.

4. The method of claim 1, 2 or 3 wherein said kinase is a viral kinase.

5. The method of any one of claims 1-4 wherein said kinase is a herpesviral kinase.

6. The method of any one of claims 1-5 wherein said kinase is from a virus selected from herpes simplex viruses, varicelloviruses, cytomegaloviruses, muromegaloviruses, roseoloviruses, lymphocryptoviruses and rhadinoviruses.

7. The method of any one of claims 1-6 wherein said kinase is from a virus selected from human herpesvirus 1 (HSV-1), human varicella zoster virus (VZV-1) or human cytomegalovirus (HCMV).

8. The method of any one of claims 1-7 wherein said kinase is selected from HCMV UL97 kinase, HSV-1 or -2 UL13 kinase, human VZV ORF47 kinase, human HHV-6 UL69 kinase, human EBV BGLF-4 kinase, human HHV-8 ORF36 kinase or kinases homologous thereto.

9. The method of any one of claims 1-8 wherein the viral kinase is encoded by:
(a) the nucleic acid sequence as shown in SEQ.ID.NO 1, SEQ.ID.NO 3 or SEQ.ID.NO 5,
(b) a nucleic acid sequence corresponding to a sequence (a) in the scope of degeneracy of the genetic code, or
(c) a nucleic acid sequence hybridizing under stringent conditions with a nucleic acid of (a) or (b).

10. The method of any one of claim 9 wherein the viral kinase has the amino acid sequence as shown in SEQ.ID.NO 2, SEQ.ID.NO 4 and SEQ.ID.NO 6.

11. The method of any one of claims 1-10 wherein said substrate is selected from ganciclovir, aciclovir and famiciclovir.

12. The method of any one of claims 1-11 wherein said target cell is a cultured eukaryotic cell.

13. The method of claim 12 wherein said target cell is a mammalian cell.

14. The method of any one of claims 1-13 wherein said phosphorylated substrate is cytotoxic for said target cell.

15. The method of any one of claims 1-14 wherein said target cell has been transformed with a vector comprising said kinase encoding nucleic acid.

16. The method of any one of claims 1-15 wherein said target cell has been infected by a virus comprising said kinase encoding nucleic acid.

17. The method of any one of claims 1-16 wherein said determining step (d) comprises a quantitative measurement of the deleterious effect mediated by said phosphorylated substrate.

18. The method of claim 17 wherein said quantitative measurement is carried out by determining signals in the culture supernatant and/or in the target cell.

19. The method of any one of claims 1-18 which is carried out as a high-throughput screening of candidate compounds for kinase-specific therapeutical drugs.

20. The method of any one of claims 1-19 further comprising the step:
(e) distinguishing between (i) noncytotoxic test compounds having kinase inhibiting properties and (ii) test compounds having kinase inhibiting properties but additionally cytotoxic side effects.

21. The method of any one of claims 1-20 wherein the effect of a test compound is determined at several different concentrations of said test compound.

22. The method of any one of claims 1-21 further comprising the determining of the effect of a test compound on a control cell.

23. The method of claim 22 wherein said control cell comprises a nucleic acid encoding an inactive variant of said kinase.

24. A reagent kit for the identification of kinase inhibitors comprising a cell containing a nucleic acid encoding a kinase and a substrate capable of being phosphorylated by said kinase and wherein said phosphorylated substrate is deleterious for said control cell.

25. Use of the reagent kit of claim 24 in a method of any one of claims 1-23.


SEQUENCE LISTING (17 sequences)

SEQ ID NO: 1 - DNA, 2124 bp, Human cytomegalovirus
atgtcctccgcacttcggtctcgggctcgctcggcctcgctcggaacgacgactcagggctgggatccgccgccattgcgtcgtcccagcagggcgcgccggcgccagtggatgcgcgaagctgcgcaggccgccgctcaagccgcggtgcaggccgcgcaggccgccgccgctcaggtcgcccaggctcacgttgatgaaaacgaggtcgtggatctgatggccgacgaggccggcggcggcgtcaccactttgaccaccctgagttccgtcagcacaaccaccgtgcttggacacgcgactttttccgcatgcgttcgaagtgacgtgatgcgtgacggagaaaaagaggacgcggcttcggacaaggagaacctgcgtcggcccgtagtgccgtccacgtcgtctcgcggcagcgccgccagcggcgacggttaccacggcttgcgctgccgcgaaacttcggccatgtggtcgttcgagtacgatcgcgacggcgacgtgaccagcgtacgccgcgctctcttcaccggcggcagcgacccctcggacagcgtgagcggcgtccgcggtggacgcaaacgcccgttgcgtccgccgttggtgtcgctggcccgcaccccgctgtgccgacgtcgtgtgggcggtgtggacgcggtgctcgaagaaaacgacgtggagctgcgcgcggaaagtcaggacagcgccgtggcatcgggcccgggccgcattccgcagccgctcagcggtagttccggggaggaatccgccacggcggtggaggccgactccacgtcacacgacgacgtgcattgcacctgttccaacgaccagatcatcaccacgtccatccgcggccttacgtgcgacccgcgtatgttcttgcgccttacgcatcccgagctctgcgagctctctatctcctacctgctggtctacgtgcccaaagaggacgatttttgccacaagatttgttatgccgtggacatgagcgacgagagctaccgcctgggccagggctccttcggcgaggtctggccgctcgatcgctatcgcgtggtcaaggtggcgcgtaagcacagcgagacggtgctcacggtctggatgtcgggcctgatccgcacgcgcgccgctggcgagcaacagcagccgccgtcgctggtgggcacgggcgtgcaccgcggtctgctcacggccacgggctgctgtctgctgcacaacgtcacggtacatcgacgtttccacacagacatgtttcatcacgaccagtggaagctggcgtgcatcgacagctaccgacgtgccttttgcacgttggccgacgctatcaaatttctcaatcaccagtgtcgtgtatgccactttgacattacacccatgaacgtgctcatcgacgtgaacccgcacaaccccagcgagatcgtgcgcgccgcgctgtgcgattacagcctcagcgagccctatccggattacaacgagcgctgtgtggccgtctttcaggagacgggtacggcgcgccgcatccccaactgctcgcaccgtctgcgcgaatgttaccaccctgctttccgacccatgccgctgcagaagctgctcatctgcgacccgcacgcgcgtttccccgtagccggcctacggcgttattgcatgtcggagctgtcggcgctgggtaacgtgctgggcttttgcctcatgcggctgttggaccggcgcggtctggacgaggtgcgcatgggcacggaggcgttgctctttaagcacgccggcgcggcctgccgcgcgttggagaacggtaagctcacgcactgctccgacgcctgtctgctcattctggcggcgcaaatgagctacggcgcctgtctcctgggcgagcatggcgccgcgctggtgtcgcacacgctgcgctttgtggaggccaagatgtcctcgtgtcgcgtacgcgcctttcgccgcttctaccacgaatgctcgcagaccatgctgcacgaatacgtcagaaagaacgtggagcgtctgttggccacgagcgacgggctgtatttatataacgcctttcggcgcaccaccagcataatctgcgaggaggaccttgacggtgactgccgccaactgttccccgagtaa

SEQ ID NO: 2 - protein, 707 aa, Human cytomegalovirus
MSSALRSRARSASLGTTTQGWDPPPLRRPSRARRRQWMREAAQAAAQAAVQAAQAAAAQVAQAHVDENEVVDLMADEAGGGVTTLTTLSSVSTTTVLGHATFSACVRSDVMRDGEKEDAASDKENLRRPVVPSTSSRGSAASGDGYHGLRCRETSAMWSFEYDRDGDVTSVRRALFTGGSDPSDSVSGVRGGRKRPLRPPLVSLARTPLCRRRVGGVDAVLEENDVELRAESQDSAVASGPGRIPQPLSGSSGEESATAVEADSTSHDDVHCTCSNDQIITTSIRGLTCDPRMFLRLTHPELCELSISYLLVYVPKEDDFCHKICYAVDMSDESYRLGQGSFGEVWPLDRYRVVKVARKHSETVLTVWMSGLIRTRAAGEQQQPPSLVGTGVHRGLLTATGCCLLHNVTVHRRFHTDMFHHDQWKLACIDSYRRAFCTLADAIKFLNHQCRVCHFDITPMNVLIDVNPHNPSEIVRAALCDYSLSEPYPDYNERCVAVFQETGTARRIPNCSHRLRECYHPAFRPMPLQKLLICDPHARFPVAGLRRYCMSELSALGNVLGFCLMRLLDRRGLDEVRMGTEALLFKHAGAACRALENGKLTHCSDACLLILAAQMSYGACLLGEHGAALVSHTLRFVEAKMSSCRVRAFRRFYHECSQTMLHEYVRKNVERLLATSDGLYLYNAFRRTTSIICEEDLDGDCRQLFPE

SEQ ID NO: 3 - DNA, 1554 bp, herpes simplex virus 1
atggatgagtcccgcagacagcgacctgctggtcatgtggcagctaacctcagcccccaaggtgcacgccaacggtccttcaaggattggctcgcatcctacgtacactccaacccccacggggcctccgggcgccccagcggcccctctctccaggacgccgccgtctcccgctcctcccacgggtcccgccaccgatccggcctccgcgagcggcttcgcgcgggactatcccgatggcgaatgagccgctcgtctcatcgccgcgcgtcccccgagacgcccggtacggcggccaaactgaaccgcccgcccctgcgcagatcccaggcggcgttaaccgcacccccctcgtccccctcgcacatcctcaccctcacgcgcatccgcaagctatgcagccccgtgttcgccatcaaccccgccctacactacacgaccctcgagatccccggggcccgaagcttcggggggtctgggggatacggtgacgtccaactgattcgcgaacataagcttgccgttaagaccataaaggaaaaggagtggtttgccgttgagctcatcgcgaccctgttggtcggggagtgcgttctacgcgccggccgcacccacaacatccgcggcttcatcgcgcccctcgggttctcgctgcaacaacgacagatagtgttccccgcgtacgacatggacctcggtaagtatatcggccaactggcgtccctgcgcacaacaaacccctcggtctcgacggccctccaccagtgcttcacggagctggcccgcgccgttgtgtttttaaacaccacctgcgggatcagccacctggatatcaagtgcgccaacatcctcgtcatgctgcggtcggacgccgtctcgctccggcgggccgtcctcgccgactttagcctcgtcaccctcaactccaactccacgatcgcccgggggcagttttgcctccaggagccggacctcaagtccccccggatgtttggcatgcccaccgccctaaccacagccaactttcacaccctggtgggtcacgggtataaccagcccccggagctgttggtgaaataccttaacaacgaacgggccgaatttaccaaccaccgcctgaagcacgacgtcgggttagcggttgacctgtacgccctgggccagacgctgctggagttggtggttagcgtgtacgtcgccccgagcctgggcgtacccgtgacccggtttcccggttaccagtattttaacaaccagctgtcgccggacttcgccctggccctgctcgcctatcgctgcgtgctgcacccagccctgtttgtcaactcggccgagaccaacacccacggcctggcgtatgacgtcccagagggcatccggcgccacctccgcaatcccaagattcggcgcgcgtttacggatcggtgtataaattaccagcacacacacaaggcgatactgtcgtcggtggcgctgcctcccgagcttaagcctctcctggtgctggtgtcccgcctgtgtcacaccaacccgtgcgcgcggcacgcgctgtcg

SEQ ID NO: 4 - protein, 518 aa, herpes simplex virus 1
MDESRRQRPAGHVAANLSPQGARQRSFKDWLASYVHSNPHGASGRPSGPSLQDAAVSRSSHGSRHRSGLRERLRAGLSRWRMSRSSHRRASPETPGTAAKLNRPPLRRSQAALTAPPSSPSHILTLTRIRKLCSPVFAINPALHYTTLEIPGARSFGGSGGYGDVQLIREHKLAVKTIKEKEWFAVELIATLLVGECVLRAGRTHNIRGFIAPLGFSLQQRQIVFPAYDMDLGKYIGQLASLRTTNPSVSTALHQCFTELARAVVFLNTTCGISHLDIKCANILVMLRSDAVSLRRAVLADFSLVTLNSNSTIARGQFCLQEPDLKSPRMFGMPTALTTANFHTLVGHGYNQPPELLVKYLNNERAEFTNHRLKHDVGLAVDLYALGQTLLELVVSVYVAPSLGVPVTRFPGYQYFNNQLSPDFALALLAYRCVLHPALFVNSAETNTHGLAYDVPEGIRRHLRNPKIRRAFTDRCINYQHTHKAILSSVALPPELKPLLVLVSRLCHTNPCARHALS

SEQ ID NO: 5 - DNA, 1530 bp, varicella zoster virus
atggatgctgacgacacaccccccaacctccaaatatctccaactgcaggacctttgcgttcccaccacaataccgacggacatgaaccaaatgcaaccgcagccgatcagcaagaacgagaatccaccaaccccacacacggatgtgtaaatcatccatgggccaatccgtcaactgcaacatgcatggaatcaccagaacgatcacaacagacaagcttatttttattaaagcacggcttaacgagagatccaatacatcaacgcgaaagggtggacgtttttccacaatttaacaaacccccatgggtttttagaatttccaaattatcccgtttaattgtacccatcttcacgctcaatgaacagttatgtttttctaaattacagattcgagatagacccaggtttgcgggacggggaacgtatgggcgtgttcatatatacccatcgtcaaaaatagctgtaaaaaccatggacagtcgtgtttttaatagagagttaattaacgcgattttagcgagtgagggttctatacgagcaggggaaaggctaggtatttctagcatagtttgccttttaggtttttcgttacaaaccaaacagctactgtttccggcatacgacatggatatggatgaatacattgttcgcctgtccagacggttgacaatacctgatcacatagacagaaaaattgcccatgtatttttagatttggctcaagcgttgacgtttttaaatcgaacgtgcggcctgacccacctagatgtgaaatgtggcaatatttttcttaacgtcgacaactttgcctcgttggaaataaccacagcagtaatcggagactatagcctagtaacattaaatacgtattccctttgtactcgagcgatatttgaagttggaaatccatcccacccggagcacgtactacgcgtaccccgggatgcatcgcagatgtcatttcgtttggtgttgagtcatggaacaaaccaaccccctgaaatcttgcttgattatattaatggaacgggccttactaaatatactggaaccttgccccaaagagttggacttgcgattgatctttatgcattgggccaagcactcttagaagttatcctgctaggacgtcttcccggacaactgcccatttcagtacatcggaccccgcattatcactactacggtcataagttatcaccagatttggcgcttgatacgctggcatatcgatgtgtcctggcgccatatatactcccatctgacatccccggggacttaaattataatccctttatacacgccggagagctgaacacccgtatttcccggaattctttacgccggatattccagtgtcacgcagtgcgttacggcgtaacgcactcaaagcttttcgaaggcatacgcattccggcctcattatacccagccactgttgttacatcgttgttgtgtcacgataattcagaaatacgctcggatcaccctttattatggcacgatcgggattggataggatcgaca

SEQ ID NO: 6 - protein, 510 aa, varicella zoster virus
MDADDTPPNLQISPTAGPLRSHHNTDGHEPNATAADQQERESTNPTHGCVNHPWANPSTATCMESPERSQQTSLFLLKHGLTRDPIHQRERVDVFPQFNKPPWVFRISKLSRLIVPIFTLNEQLCFSKLQIRDRPRFAGRGTYGRVHIYPSSKIAVKTMDSRVFNRELINAILASEGSIRAGERLGISSIVCLLGFSLQTKQLLFPAYDMDMDEYIVRLSRRLTIPDHIDRKIAHVFLDLAQALTFLNRTCGLTHLDVKCGNIFLNVDNFASLEITTAVIGDYSLVTLNTYSLCTRAIFEVGNPSHPEHVLRVPRDASQMSFRLVLSHGTNQPPEILLDYINGTGLTKYTGTLPQRVGLAIDLYALGQALLEVILLGRLPGQLPISVHRTPHYHYYGHKLSPDLALDTLAYRCVLAPYILPSDIPGDLNYNPFIHAGELNTRISRNSLRRIFQCHAVRYGVTHSKLFEGIRIPASLYPATVVTSLLCHDNSEIRSDHPLLWHDRDWIGST

SEQ ID NO: 7 - DNA, 31 bp, Artificial Sequence
  PCR primer
tagtagatctatgtcctccgcacttcggtct

SEQ ID NO: 8 - DNA, 31 bp, Artificial Sequence
  PCR primer
tagtgtcgacttactcggggaacagttggcg

SEQ ID NO: 9 - DNA, 27 bp, Artificial Sequence
  PCR primer
catgccatgggcatgtcctccgcactt

SEQ ID NO: 10 - DNA, 24 bp, Artificial Sequence
  PCR primer
ccgctcgagctcggggaacagttg

SEQ ID NO: 11 - DNA, 27 bp, Artificial Sequence
  PCR primer
cccgaattcatgtcctccgcacttcgg

SEQ ID NO: 12 - DNA, 51 bp, Artificial Sequence
  PCR primer
ccgctcgagttacttgtcgtcatcgtctttgtagtcctcggggaacagttg

SEQ ID NO: 13 - DNA, 27 bp, Artificial Sequence
  PCR primer
cccgaattcatgtcctccgcacttcgg

SEQ ID NO: 14 - DNA, 60 bp, Artificial Sequence
  PCR primer
ccgctcgagttacttgcccagccggttcatctcgatgtcggtgtactcggggaacagttg

SEQ ID NO: 15 - DNA, 54 bp, Artificial Sequence
  PCR primer
ccgctcgagttaagcgtaatctggaacatcgtatgggtactcggggaacagttg

SEQ ID NO: 16 - DNA, 27 bp, Artificial Sequence
  PCR primer
cttacgcgccaccatgaccacgcgata

SEQ ID NO: 17 - protein, 16 aa, Artificial Sequence
  n-terminal peptide of HCMV UL97 kinase
MSSALRSRARSASLGT